(19) United States
(12) Patent Application Publication
(10) Pub. No.: US 2010/0030480 A1
(43) Pub. Date: Feb. 4, 2010

(54) DEVICE AND METHOD FOR EXAMINATION AND EVALUATION OF A BIOLOGICAL ACTIVE AND/OR BIOLOGICAL ACTIVATABLE SUBSTANCE

(76) Inventor: Jahn Wolfgang, Landshut (DE)

Correspondence Address: LADAS & PARRY LLP, 224 SOUTH MICHIGAN AVENUE, SUITE 1600, CHICAGO, IL, 60604, US

(21) Appl. No.: 12/299,642

(22) PCT Filed: May 4, 2007
(86) PCT No.: PCT/EP2007/003976

§ 371 Date: Oct. 1, 2009

(30) Foreign Application Priority Data
May 5, 2006 (DE) 20 2006 012 071.8

(51) Int. Cl. G01N 21/64; G01N 21/00; G06F 19/00; G01J 3/00; A61B 5/00

(52) U.S. Cl. 702/19; 356/317; 356/326; 356/51; 600/310

(57) ABSTRACT

The invention relates to a device and method for optical examination and for evaluation of a biological active and/or biological activatable substance (2) by use of infra-red, visible or ultra-violet light, at which the examined, respectively evaluated substance is optically stimulated, so that the substance either emits light (in particular fluorescence analysis), or modifies the wavelength of the used light (in particular raman analysis), whereas the device comprises:
An electrically driven light source (3),
an optical light guide (4), by which the light emitted by the light source (3), is transferable as radiation to the substance being examined (2),
an optical or opto-electronical sensor device (5) dedicated to the substance being examined (2), to receive the light emitted or modified by the biological active and/or biological activatable substance,
an optical or opto-electronical spectrometer device (6) dedicated to the sensor device (5), by which the light emitted or modified by the substance (2) is measured regarding to light intensity at not less than one frequency, or regarding to frequency shift, whereas a corresponding measurement signal is passed to an analysis circuitry assigned to the spectrometer device (6), and
the device featuring a measurement surface (11), which in shape and dimensions fits to the biological active and/or biological activatable substance (2) being examined.

The invention relates to a device and a method for optical examination and for evaluation of a biological active and/or biological activatable substance, in particular of human or animal body tissue, by use of infra-red, visible or ultra-violet light, at which the examined substance is optically stimulated, so that the substance either emits light (fluorescence analysis), or modifies the wavelength of the used light (raman analysis).

Human (or animal) metabolism is the sum of all life sustaining biochemical and biophysical processes. Metabolism differs from person to person, and it changes over lifetime, furthermore it depends on many other factors like physical or psychic stress or illness. By use of metabolism analysis it is possible to achieve a comprehensive knowledge about the current health condition of the human organism. Up to now, metabolism analysis only uses the findings of different single examination methods, such methods being expensive and timeconsuming. These methods are, in particular, laboratory analyses of organ activities, of the immune system, of the endocrinal system, of the lipometabolism, of the mineral metabolism, etc.

So far, metabolism analysis still requires the taking and analysis of a blood sample, with the blood sample being sent to a laboratory for analysis. Apart from the costs, there are other disadvantages. Because of the transport and the long duration of the analysis, aberrances in the blood sample can occur (e.g. oxidation effects, freezing effects), which affect the result of the examination. Moreover, taking a blood sample is quite inconvenient for the patient, and sometimes it even bears some dangers. In addition, various spectrometer devices for optical examinations of the fluorescence properties of biological tissue samples are known from prior art. Proteins of the biological substance of various species are intrinsic fluorescent, e.g. porphyrin, aromatic amino acids like tryptophan, tyrosine, phenyialanine and the like. An unfavorable fact is that the biological tissue samples have to be taken from the test subject and then, before executing an optical examination, have to be placed on a sample carrier or in a bulb. Prior art examination methods take place in a Laboratory and are not suitable for immediate and quick examinations of the test person on location respectively in the working- or recreational environment. Furthermore, these methods require invasive action on the respective part of the body.

Object of this invention is to provide a device and a method for optical examination and in particular for evaluation of a biological active and/or biological activatable substance, in particular of human or animal body tissue, especially with regard to the substances of the cellular metabolism; said device and method facilitating a simple (i.e. to be carried out by the test subject himself), instant and fast examination of the test subject on location respectively in the working- or recreational environment, whereas, more than a bare examination, said device and method ensure or at least offer a medically sound and reproducible overall evaluation of the organism belonging to the examined substance.

This object is solved by the device as described in claim 1 and the method as described in claim 12.

The device according to the invention features an electrically driven light source, an optical light guide, by which the light emitted by the light source is transferable as radiation to the substance being examined, an optical or opto-electronical sensor device dedicated to the substance being examined, to receive the light emitted or modified by the biological active and/or biological activatable substance, an optical or opto-electronical spectrometer device dedicated to the sensor device, by which the light emitted or modified by the substance is measured regarding to light intensity at not less than one frequency, or regarding to frequency shift, whereas a corresponding measurement signal is passed to an analysis circuitry assigned to the spectrometer device, and the device featuring a measurement surface, which in shape and dimensions fits to the biological active and/or biological activatable substance being examined.

According to the principle of the invention, the biological active and/or biological activatable substance is a biological tissue, i.e. a tissue of human, plant or animal origin. Basically, an application on other, not necessarily living matter is also imaginable, for example for measuring biologically relevant factors in a soil substance or humus, or other substances of the category “biological activatable” like nutrient solutions or sugar solutions et cetera.

The present invention allows a non-invasive optical examination of a biological active and/or biological activatable substance, in particular tissue material of a test subject; no tissue samples have to be taken or have to be inserted or clamped into measurement probes or the like. Because of the fact that the light emission segment of the optical light guide and the sensor device form a structural entity, with said entity comprising a measurement surface on its upper or outer surface, with the measurement surface matched in shape and dimensions to the human or animal biological tissue being examined, optical examination of the substance can easily be accomplished by bringing the measurement surface to a suitable position on the skin surface of the test subject with skin contact or not, and by keeping it in place for the duration of the measurement process, typically for a few seconds. The invention allows a immediate and quick examination of the test person on location respectively in the working- or recreational environment, with the examination being executed by the test person.

The device according to the invention allows an examination, and particularly an instant subsequent evaluation of the biological tissue within seconds, whereas an instant presentation, respectively information on the current total-health-state, based on this non-invasive metabolism analysis, can be assured. The device respectively method offers a depiction of a test subject's current total-health-state, which can provide important and essential details on medically relevant factors regarding the patient to a physician.

The device according to the invention measures autofluorescence of the body's endogenous interstitial metabolism substances, activated by irradiation, particularly by short wave UV radiation or by near range UV radiation. The advantages of the device according to the invention are, in addition to the bloodless, non-invasive analysis method, primarily the only few seconds lasting examination and analysis, thus offering a cost-efficient monitoring of the metabolic regulation without affecting the patient. Auto- or primary fluorescence of cells, tissues and substances is based on the stimulation of endogenous fluorophores. Important endogenous fluorophores are e.g. tryptophan, coenzymes NADH, NADPH and flavins, as well as porphyrins containing zinc and metal free porphyrins. In medical science, this phenomenon can be used for diagnostics, for example for early diagnosis of malignant cell mutations of the skin and in the mucous membranes of hollow organs. Measurement of the metabolism substances dissolved in the interstitium is medically relevant and quite significant. The term “interstitium” refers to the liquid filled space between cells, which is filled with connective tissue, nerves and blood vessels, and is in constant contact with the cells, the blood vessels and the lymphatic system. In the interstitium, transport of nutriments to the cells and transport of metabolites from the cells take place as well as transport of molecular cues between cells. Changes in cell metabolism or disturbed cell functions affect the composition of the interstitial fluid in a specific way. Thus, the interstitium is best suited for both measuring temporary changes of metabolism caused by physical or psychical stress and metabolism disorders caused by organic diseases or ailments.

The measured autofluorescence values detected with the device according to the invention are combined in a specific way, and so they offer information about existing metabolism disorders, the influence of stress or other regulation disorders; thus a picture of the current state of health of the test subject is presented. The established parameters allow the following conclusions about the current state of health: Metabolic rate regulation, protection against acidity, immune defence, condition of connective tissue, regulation of inflammatory processes, protection against oxidative stress, mental resilience, allergic activation, protection against infective processes, cellular neoplasm processes, cellular degradation, and current micro-nutritional requirements. The invention finally permits to make reproducible, medically sound statements about the general metabolism activity, its regulation and the influence of inflammations, allergies, viral and/or bacterial infecntions in a comparatively straight-forward manner. Furthermore, with the invention, another field of application for light-induced autofluorescence is opened.

In the following, the conclusions attainable with the device/with the method will be described. Protection against acidity: Acidity occurs where metabolism takes place intensively and when acids, produced during the metabolism process, cannot be decomposed sufficiently. The whole metabolism is oriented towards the production of acids, all intermediate metabolic products are acids which have to be buffered respectively bound in different ways. The so-called acidity takes place in particular in the intestine (during metabolism). An excess of acids, accompanied by a shift of the pH-level, causes primarily disturbances of the enzyme activity, which in turn leads to metabolism disorders, to severest inflammations, to a weak immune system and to diseases. Values tending towards the “red zone” often indicate the risk of acidity, which can lead e.g. to permanent inflammations, bad skin appearance, diarrhoea, obstipation, up to autoimmune diseases. Immune defence: This parameter reveals how intensive the organism raises antibodies to resist antigenes. The higher the measured values are in the “red zone”, the higher is the danger of overstraining the immune system, i.e. the organism is no longer able to defend itself, so various diseases can arise. Metabolic rate regulation: This parameter reveals how the organism is able to metabolize and to exploit nutriments and to keep up the own body functions (growth, temperature regulation, regeneration, immune response, etc.) The higher the values measured by the invention are in the “red zone”, the higher is the likelihood of capability constraints, respectively constraints of all body- and immune functions. Condition of connective tissue: Connective tissue is everything that keeps our body together, i.e. Collagen and elastic fibres, ligaments, sinews, and subcutis fibres. The higher the corresponding values are in the “red zone”, the higher is the risk of a disturbance of the maintenance of a certain percentage of elastic and collagenous parts necessary for muscle tone and normal reaction of the muscles. This can lead to loose tissue, impairment of mobility (functions of joints), and even to diseases like arthrosis, arthritis, etc. Regulation of inflammatory processes: Inflammatory processes are a part of life which take place permanently; These processes must take place, because they belong to normal regulation of metabolism, immune system and enzyme functions (except for chronic processes).The higher the corresponding values are in the “red zone”, the higher is the likelihood of chronic inflammations disturbing life functions. Protection against oxidative stress: Oxidative protection covers all substances being able to reduce oxidative stress (free radicals), these are the so-called antioxidants. Existence of the organism is dependent from oxygen, it allows to gain energy from nutrition to maintain all body functions. On the other hand oxygen also has destructive effects, because during metabolism a formation of oxygen species (so-called radicals) is possible, which are highly aggressive and destructive. Usually, a healthy organism can cope with this strain, but this natural strain can under certain circumstances be increased, such circumstances being an unhealthy way of life and health critical life situations like physic and psychic pressure to perform, consumption of stimulants and pharmaceuticals, toxic environment influences, changed alimentary habits and the like. This leads to an excess of free radicals with no adequate protection, thus producing oxidative stress. Meanwhile it has been found that 70% of all diseases are caused by oxidative stress. Thus, a permanent protection respectively an adequate supply with antioxidants is a major requirement for the existence of every organism. The higher the corresponding values are in the “red zone”, the higher is the risk for oxidative stress, because no adequate supply with antioxidants exists. Mental resilience: This is the ability to think, to transform the thinking into will, and to control the will that way, that the body is directed by the will.

Allergic activation: Allergic activation in a healthy context is the fight of the immune system against all foreign substances being produced in the body or being imported. Comparable to inflammations, allergies are essential permanent processes in the body. However, if an overreaction occurs, the well-known allergic symptoms arise. The higher the measured values are in the “red zone”, the higher is the overreaction of the immune system; in other words: the faster the overreaction decays, the better the immune regulation works. Protection against infective processes: This does not mean the strength of protection against infections, because, as inflammations and allergic reactions, infections have to occur permanently to preserve viability. The shorter the duration of infectious symptoms, the higher is the protection, i.e. the early stemming, so that the body is not unnecessarily high strained by the infection. The worse the measured values are, the higher is the risk that an infectious process develops into a severe infection. Cellular neoplasm processes: These contain many physiological and pathological processes, thus the method according to the invention merely offers a rough orientation whether these bad values indicate malignant neoplasm or only a increased cell reproduction within the scope of regeneration processes. It is important to look at neoplasm always in connection with cell degradation. If the balance between neoplasm and cell degradation is disturbed, the cause must be examined by a physician. Cellular degradation: This parameter also only gives a rough orientation, it indicates whether cell degradation is taking place at a high rate. As above, it is important to look at neoplasm always in connection with cell degradation. General performance: This is a summarizing statement combining factors like e.g. immune defence, metabolic rate regulation, oxidative stress, etc., as well as recuperation by sleep and the like. Training condition: The ability of the organism to withstand strain caused by radicals. The more the values are in the “red zone”, the less the organism is protected against radicals, which in turn means it is less resilient.

The device according to the invention works the following way: Application of a human or animal body part onto the sensor device stimulates various substances with fluorescending properties in the tissue liquid by light pulses emitted by the light source; as a reaction these substances start emitting fluorescence light of certain wavelength, with this fluorescence light being analyzed computer-aided, and the fluorescence values of these analyzed parameters being calculated in relation to each other.

From optical examination or analysis of the biological active and/or biological activatable substance different specific metabolism parameters can be calculated, these specific metabolism parameters comprising cell division activity and/or metabolic activity and/or activity of biosynthesis and/or regulation of immune system and/or regulation of inflammatory processes and/or metabolic rate regulation and or energy consumption for cell division activity and/or regular and irregular neoplasm and/or inflammatory regular neoplasm and/or health threat risk parameters and/or risk parameter of activated neoplasm and/or complex trait of metabolism and/or redox equivalent and/or mental stability parameter.

In accordance with the principle of the invention, at least a portion of the optical light guide and the sensor device can be placed in a housing, said housing comprising a contact area on or in the upper portion of the housing, with the contact area including the measurement surface, or at least partially correlating with the measurement surface, on said contact area a part of the body of the substance being examined, in particular a hand, a forearm, a leg or a foot is applied, whereas an alignment device is assigned to the contact area on or in the upper portion of the housing for aligning the body part of the test subject with the contact area.

In a preferred embodiment of the invention the optical light guide can feature a light emission segment, which forms, together with the sensor device, a structural entity, said entity comprising the measurement surface on its upper or outer surface. Hereby, the alignment device can feature a recess or a bulge on or in the upper portion of the housing, the width of said alignment device approximating the width of a middle finger.

Following the principle of the invention, the optical examination is carried out on an unclothed or easily uncoverable body portion of the skin of the hand, the arm, the foot, the leg, the face, or the neck- and upper shoulder area of the test subject. According to the invention, the measurement surface is of almost convex shape following the contours of such a body part, for example a slightly clenched hand. Preferably the light source, the optical light guide, the sensor device and the spectrometer are placed in a housing, said housing comprising a contact area on or in the upper portion of the housing, with the contact area including the measurement surface, or at least partially correlating with the measurement surface, on said contact area a part of the body of the substance being examined, in particular a hand, a forearm, a leg or a foot is applied, whereas an alignment device is assigned to the contact area on or in the upper portion of the housing for aligning the body part of the test subject with the contact area.

In another preferred embodiment of the invention the measurement surface is curved strongly convex with the shape following approximately the contours of a clenched hand. In this case, the measurement surface can be designed as part of the surface of a handle bar or a steering wheel, or a control column, or a control stick, or as other steering- or control device of a land-, water-, air- or space vehicle. The benefit of this embodiment is in particular to examine the guider of a vehicle, for example a long distance lorry driver, a ship steersman or a pilot, who possesses a particular responsibility regarding vigilance and concentration, during his duty medically and biologically for taking adequate countermeasures in case of disturbances (e.g. vehicle driver's fatigue). With this invention it is possible to implement a safety device protecting the driver from falling asleep, thus preventing accidents of that kind.

In another preferred embodiment of the invention, an image displaying device is placed on the measurement surface or adjacent to it, on which the measurement results are displayed as well as a menu-driven software program for controlling the device according to the invention.

The optical spectrometer device according to the invention can comprise a grating spectrometer or a fourier transform spectrometer or a filter spectrometer. A grating spectrometer uses diffraction of light rays on a grating for dispersion. Sampling of the spectrum is accomplished by either using a rotating grating, by which different wavelengths are sampled temporally offset by a single-channeled detector, or by using a static grating with sampling of the spectrum in the desired wavelength range accomplished by a multichannel detector. A fourier transform spectrometer is characterized by an interferometric filter being varied, the variation being recorded as a so-called interferogram. The desired spectrum is obtained by fourier transformation of of the interferogram in the desired wave vector area. Filter spectrometers are based on a filter screen selecting the single wavelengths. In general, filter spectrometers have the following two disadvantages: First, the production of a good filter screen can only be accomplished by using very advanced coating techniques. Second, the light being analyzed crosses the filter screen, which causes a loss of light, and so only a bad signal-to-noise-ratio can be achieved. Because of a comparatively simple construction and a very good wavelength resolution the grating spectrometer is first choice for use in the embodiment according to the invention.

A downsizing of the device according to the invention can be carried out when in another embodiment the optical or opto-electronical spectrometer device assigned to the sensor device features a semiconductor sensor apparatus with at least an avalanche photo diode with a band gap corresponding to the frequency or frequency shift to be measured. In this connection, the semiconductor sensor apparatus can feature an adjustable and/or variable frequency gap, with regard to the measurement frequency.

The method according to the invention comprises the following steps:
Irradiation of the substance being examined with light emitted by the light source, said light being transferred to the substance via optical light guide,
receiving the light emitted or modified by the biological active and/or biological activatable substance using an optical or opto-electronical sensor device dedicated to the substance being examined,
measurement of the light emitted or modified by the substance regarding to light intensity at not less than one frequency, or regarding to frequency shift, by use of an optical or opto-electronical spectrometer device dedicated to the sensor device, and
passing of a corresponding measurement signal to an analysis circuitry.

Hereby, the biological active and/or biological activatable substance is a biological tissue, i.e. a tissue of human, plant or animal origin.

Following the principle of the method according to the invention it is intended that by application of the biological active and/or biological activatable substance onto the sensor device, different fluorescending components n, m (n, m=a, b, c, d, . . . ) of the substance will be activated by a light impulse emitted by the light source, whereupon these components (a, b, c, d, . . . ) start emitting light signals of certain wavelengths and certain intensities, with these light signals being measured.

Hereby, from the measured intensities (Iₐ, I_{b}, I_{c}, I_{d}) of the light signals, multiple, in fact at least three or four, in particular at least five or six different metabolic parameters Sₙ (with n=a, b, c, d, . . . ) are being calculated, whereas the following applies:

Sₙ=F (Iₙ, λₙ)×G (Iₘ, λₘ)

with F and G being two different mathematical functions of intensities and wavelengths, with n, m=a, b, c, d . . . , and n≠m,

and the correlation, respectively the mathematical conjunction X of the functions F and G in the most basic form being a product or a sum, in the following shape:

Sₙ=Π[F (Iₙ, λₙ)G (Iₘ, λₘ)] or

Sₙ=Σ[F (Iₙ, πₙ)G (Iₘ, λₘ)],

with n, m=a, b, c, d, . . . , and n≠m, or n=m.

The components n, m with their respective measured emission wavelengths (λₙ, λₘ) and their measured intensities (Iₙ, Iₘ) in particular exhibit the following biochemical substances:

a=ATP (Adenosine triphosphate), and/or

b=GTP (Guanosine triphosphate), and/or

c=FAD (Flavinadenindinucleotide), and/or

d=NADH (Nicotinamide adenine dinucleotide reduced), and/or

e=NADP (Nicotinamide adenine dinucleotide phosphate), and/or

f=Kynurenine, and/or

g=Orotic acid, and/or

h=Thromboxane, and/or

i=Tryptophan

The fluorescence intensities (Iₙ, Iₘ) in the wavelength range (λₙ, λₘ) from 287 to 800 nm, preferably from 340 to 600 nm are measured for the components n, m=a, b, c, d, . . . .

When measurement of the fluorescence intensities (Iₙ, Iₘ) occurs at a defined moment (tₙ, tₘ) and/or in defined intervals (Δtₙ, Δtₙ), these progression measurements can unveil different control- and regulation processes, whereas the following applies: n≠m, tₙ≠tₘ, Δtₙ1≢Δtₘ.

If, at a defined moment (tₙ, tₘ) of the measurement, a psychic or physical stress is applied on the patient, and the fluorescence intensities (Iₙ, Iₘ) are being measured several times before and after stress application, the metabolic regulation can be determined.

From optical examination or evaluation of a biological active and/or biological activatable substance and from measurement of the metabolic parameters Sₙ (with n=a, b, c, d, . . . ), various different specific state variables Zₙ are being calculated and graphically depicted as mathematical functions of the metabolic parameters Sₙ, whereas these specific state variables Zₙ comprise the following:

α=Cell division activity, and/or

β=Metabolic activity, and/or

γ=Activity of biosynthesis, and/or

δ=Regulation of immune system, and/or

ε=Inflammations, and/or

ζ=Metabolic rate, and/or

η=Energy consumption for cell division activity, and/or

θ=Regular and irregular neoplasm, and/or

ξ=Inflammatory regular neoplasm, and/or

ρ=Health threat risk parameters, and/or

σ=Activated neoplasm, and/or

φ=Complex trait of metabolism, and/or

χ=Redox equivalent, and/or

ψ=Mental stability parameter.

The method according to the invention can be utilized in particular for non-invasive examination of control- and regulation processes of human and animal metabolism, and/or for diagnosis of diseases and for preventive examinations, and/or for routine examinations of occupational groups and athletes with a high exposure to physical and psychic stress, and/or for therapy control, and/or for progress of dialysis- and apheresis treatment and for determining the demand for antioxidants.

In a preferred embodiment of the method according to the invention, fluorescence intensities in the wavelength range from about 287 nm to about 800 nm, preferably from 340 nm to 600 nm, are being measured, in particular for metabolic relevant substances with predetermined emission wavelengths, by preference ATP, GTP, FAD, NADH, NADP, kynurenine, orotic acid, thromboxane and tryptophan.

Preferably, the biological active components showing autofluorescence are being activated to emit by application of light with an excitation wavelength of 287 nm to 340 nm, preferably 340 nm, onto the cellular and intercellular area.

Preferred embodiments of the invention are cited in the subsequent claims.

Further advantages and efficacies are revealed in the following description of embodiments with drawings.

It shows:

FIG. 1 a schematic view of the device according to the invention, in a first embodiment example;

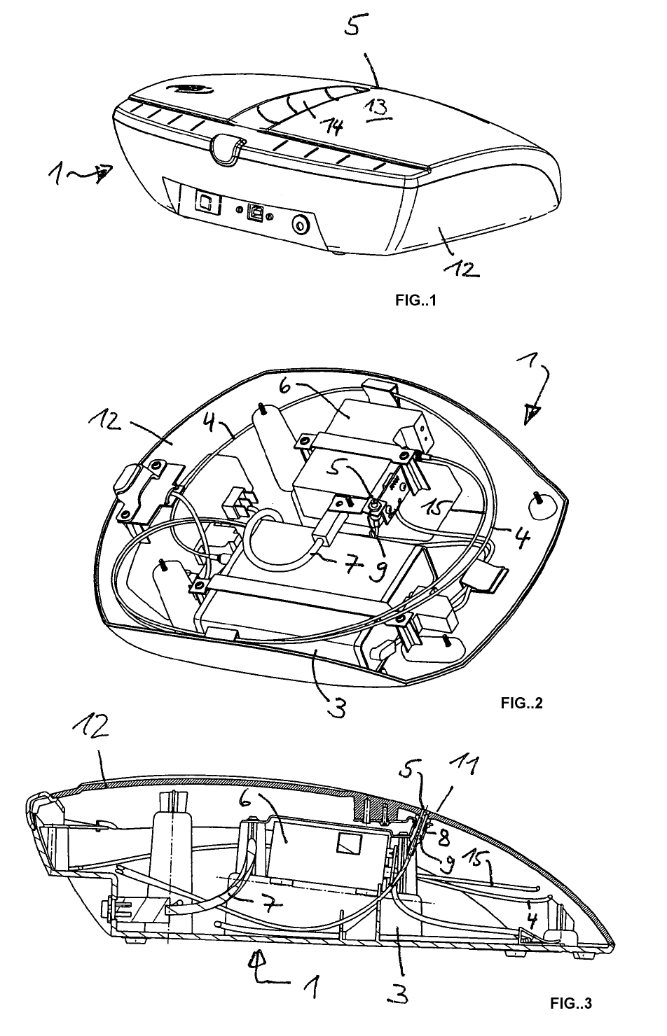

FIG. 2 an isometric view of the first embodiment example;

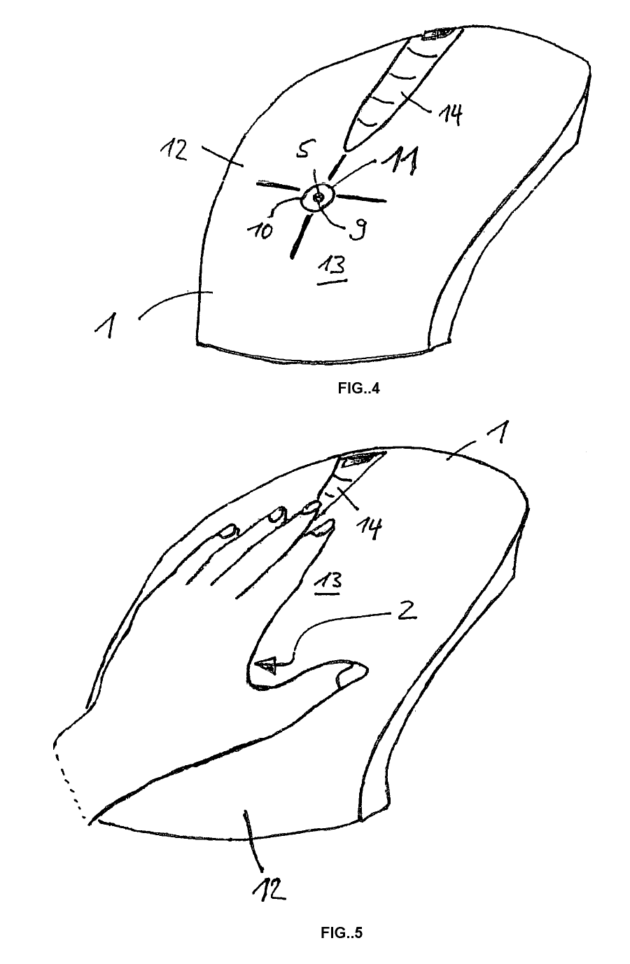

FIG. 3 a cross-sectional view of the first embodiment example;

FIG. 4 a total view of the first embodiment example;

FIG. 5 a total view of the first embodiment example with applied hand;

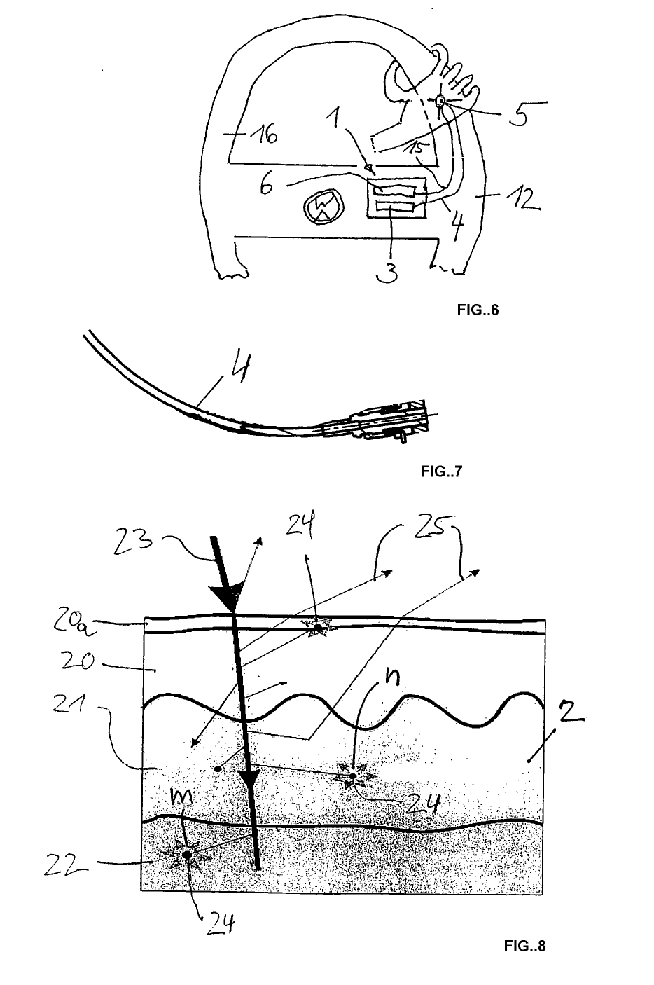

FIG. 6 a total view of a second embodiment example;

FIG. 7 a schematic cross-sectional view of the optical light guide with the light emission segment;

FIG. 8 a schematic cross-sectional view of the human skin surface for explanation of the measurement method according to the invention;

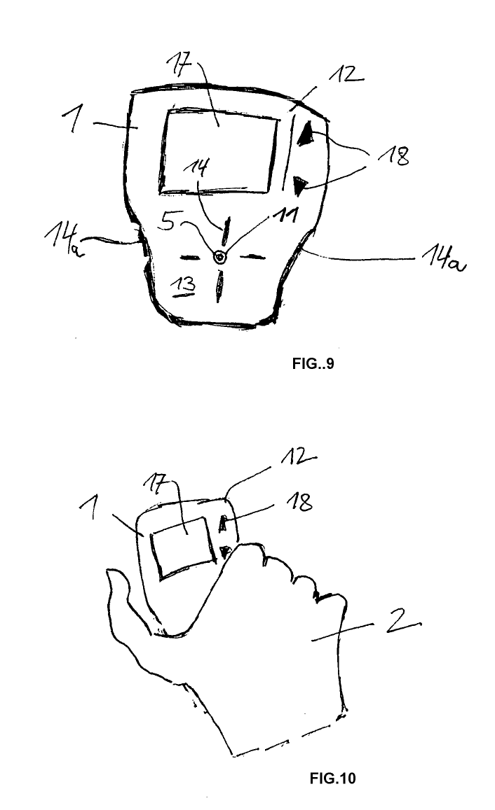

FIG. 9 a total view of a third embodiment example;

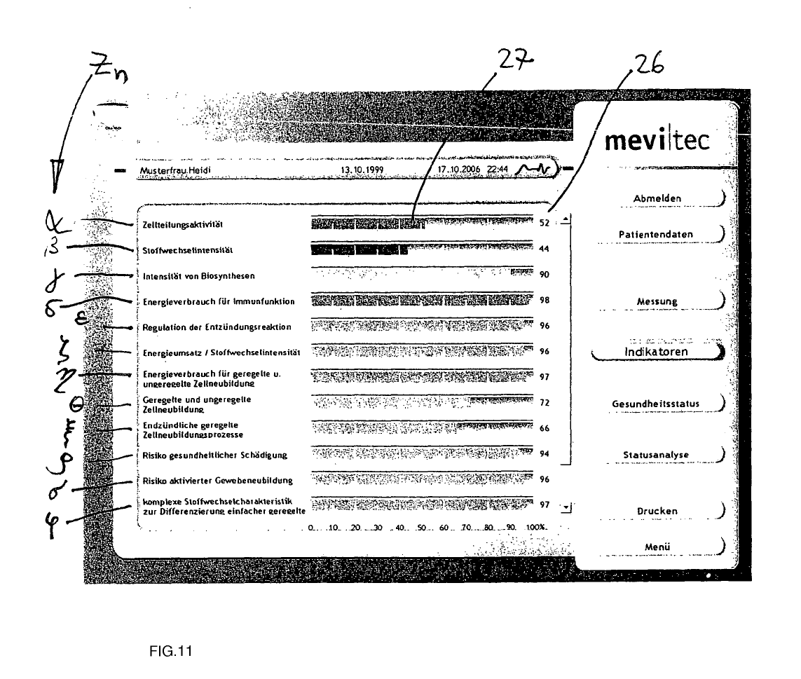

FIG. 10 a total view of the third embodiment example with applied hand; and

FIG. 11 a schematic screen view of the specific state variables Zₙ, established with the method according to the invention.

In the figures embodiment examples of the invention are displayed, whereas similar reference numbers mark similar parts.

The figures show a device 1 for optical examination or evaluation of a biological active and/or activatable substance 2 by use of infra-red, visible or ultra-violet light, at which the examined, respectively evaluated substance 2 is optically stimulated, so that the substance 2 either emits light (in particular fluorescence analysis), or modifies the wavelength of the used light (in particular raman analysis),

The device 1 comprises an electrically driven light source 3, an optical light guide 4, by which the light emitted by the light source 3 is transferable as radiation to the substance being examined 2, an optical or opto-electronical sensor device 5 dedicated to the substance being examined 2 to receive the light emitted or modified by the biological active and/or biological activatable substance, an optical or opto-electronical spectrometer device 6 dedicated to the sensor device 5, by which the light emitted or modified by the substance 2 and coupled out via a light wave conductor 15, is measured regarding to light intensity at not less than one frequency, or regarding to frequency shift, whereas a corresponding measurement signal is passed via a USB-link 7 to a (not depicted) analysis circuitry of an external computer. According to the invention the light emission segment 8 of the optical light guide 4 and the sensor device 5 together form a structural entity 9; said entity 9 comprising a measurement surface 11 on its upper or outer surface 10, said measurement surface 11 fitting in shape and dimension to the human or animal biological tissue 2 being examined.

On the first embodiment as shown in the FIGS. 1 to 5, the measurement surface 11 is of almost convex shape following approximately the contours of a slightly clenched hand 2. Here, the light source 3, the optical light guide 4, the sensor device 5 and the spectrometer device 6 are placed in a housing 12, said housing comprising a contact area 13 on or in the upper portion of the housing 12, with the contact area 13 including the measurement surface 11, or at least partially correlating with the measurement surface 11, on said contact area 13 the human or animal tissue to be examined 2, in particular a hand, is applied, whereas an alignment device 14 is assigned to the contact area 13 on or in the upper portion of the housing for aligning the body part of the test subject with the contact area 13. In the depicted embodiment, the alignment device 14 features a recess on or in the upper portion of the housing, the width of said alignment device 14 approximating the width of a middle finger.

On the second embodiment as shown in FIG. 6, the measurement surface 11 is curved strongly convex with the shape following approximately the contours of a clenched hand. In this embodiment, the measurement surface 11 can be designed as a part of the surface of a car steering wheel 16, or a control column or a control stick, or as other steering- or control device of a land-, water-, air- or space vehicle. The car steering wheel 16 serves as housing 12 of the device 1 according to the invention.

On the third embodiment as shown in FIGS. 9 and 10, the measurement surface 11 is shaped approximately to the contours of a slightly clenched hand. On or in the upper portion of the housing 12, a contact area 13 including the measurement surface 11, or at least partially correlating with the measurement surface 11 is established, on said contact area 13 the human or animal tissue to be examined 2, in particular a hand, is applied, whereas an alignment device 14 is assigned to the contact area 13 on or in the upper portion of the housing for aligning the body part of the test subject with the contact area 13. Additionally, the edges 14a of the housing are rounded, and they also serve as guides for the hand 2. On the third embodiment as shown in FIGS. 9 and 10, the housing 12 is, compared to FIGS. 1 to 3, of smaller dimensions; in this embodiment the light source 3 and the spectrometer device 6 can be placed in a separate part, whereas the optical and opto-electronical components placed in that part are connected to the sensor device 5 placed in the housing 12 via the optical light guide 4. In these embodiment, an image displaying device 17 is placed on the measurement surface 11 or adjacent to it; this displaying device being for example a miniature screen 17 integrated into the housing 12. Function buttons 18 are positioned next to the image displaying device 17 to control the measurement method displayed on the screen.

FIG. 8 shows schematically the anatomy of the skin for explanation of the analysis method according to the invention. The human (or animal) skin for example consists of three interconnected different layers, whereas the outer layer 20 is called epidermis, the middle layer 21 is termed corium (dermis), and the lower layer 22 is called subcutis. The epidermis is the outer layer of the skin,it possesses no blood vessels, and it is divided into sublayers. The outer sublayer is the “horny layer” (stratum corneum), on which the light guide is placed. On this layer 20a about 5 to 7% of the streaming light are re-emitted. The epidermis 20 is followed by the corium (dermis) 21. The corium 20 gives the skin its tensile strength and its deformability. The next layer is the subcutis 22, which is loosely connected to the muscle fibres beneath. The subcutis consists of connective tissue and serves, amongst others, as a fat reservoir. Between epidermis 20 and corium 21, a network of arterioles and venules ensures the supply of the skin cells. The entering light 23 is dispersed on the cellular structures of the various layers and is absorbed by skin pigments, bilirubin, oxyhaemoglobin and deoxyhaemoglobin. Thus, skin tissue consists of different cell types and is nerved by tiny muscle-free blood vessels (blood capillary), which are connected by venules (small veins) and arterioles (small arteries). The blood capillary enable the metabolite exchange between blood and tissue (e.g. electrolytes, O₂, CO₂) . Arteries, veins and capillaries form the transport system of the body that provides the organs and the tissue with oxygen and other nutriments and facilitates the outflow of deoxygenated blood and metabolic products. The method according to the invention is based on the measurement of autofluorescence of endogenous interstitial metabolism substances 24, activated by irradiation 23, particularly by short wave UV radiation or by near range UV radiation. Due to this auto- or primary fluorescence of cells, substances and tissues, the activated endogenous fluorophores re-emit specific light beams 25 with different wavelengths, which are detected by the sensor device according to the invention.

Different fluorescending components n, m (n, m=a, b, c, d, . . . ) of the substance 2 are activated by a light impulse 23 emitted by the light source, whereupon these components (a, b, c, d, . . . ) start emitting light signals of certain wavelengths and certain intensities, with these light signals being measured. Hereby, from the measured intensities (Iₐ, I_{b}, I_{c}, I_{d}) of the light signals, multiple, in fact at least three or four, in particular at least five or six different metabolic parameters Sₙ (with n=a, b, c, d, . . . ) are being calculated, whereas the following applies: Sₙ=F (Iₙ, λₙ)×G (Iₘ, λₘ)

with F and G being two different mathematical functions of intensities and wavelengths, with n, m=a, b, c, d . . . , and n≠m, and the correlation, respectively the mathematical conjunction X of the functions F and G in the most basic form being a product or a sum, in the following shape:

Sₙ=Π[F (Iₙ, λₙ)G (Iₘ, λₘ)] or

Sₙ=Σ[F (Iₙ, λₙ)G (Iₘ, λₘ)],

with n, m=a, b, c, d, . . . , and n≠m, or n=m.

The components n, m with their respective measured emission wavelengths (λₙ, λₘ) and their measured intensities (Iₙ, Iₘ) in particular exhibit the following biochemical substances: a=ATP (Adenosine triphosphate), b=GTP (Guanosine triphosphate), c=FAD (Flavinadenindinucleotide), d=NADH (Nicotinamide adenine dinucleotide reduced), e=NADP (Nicotinamide adenine dinucleotide phosphate), f=Kynurenine, g=Orotic acid, h=Thromboxane, i=Tryptophan.

From optical examination or evaluation of a biological active and/or biological activatable substance and from measurement of the metabolic parameters Sₙ (with n=a, b, c, d, . . . ), various different specific state variables Zₙ are being calculated and graphically depicted as mathematical functions of the metabolic parameters Sₙ, whereas these specific state variables Zₙ comprise the following (see FIG. 11):

α=Cell division activity: Informs whether regular or irregular neoplasm activity occurs, and in particular about the degree of activity of cell neoplasm (normal or disturbed).

β=Metabolic activity: Informs about the state of metabolic regulation, i.e. about composition and degradation respectively the utilisation of food and nutriments. In case of disturbances it is possible that fermentation is taking place, or gluconeogenesis is mainly based on proteolysis. The accumulation of various metabolic products can endanger the whole organism and lead to various diseases.

γ=Activity of biosynthesis: Includes all synthesis processes running in life, i.e. the anabolism with a constant modification and production of cells. A decreased activity leads to disturbances regarding maintenance of the body substance, e.g. wound healing, immune reactivation, etc. This means all necessary processes for renewal of the body structure.

δ=Regulation of immune system: Informs about how effective the immune defence works against pathogens of all kinds. ε=Inflammations: It is measured whether inflammatory processes take place in the body.

ζ=Metabolic rate: It is measured how well the energy derived from nutrition is used, consumed respectively converted, e.g. for growth, thermal balance, organ activities etc. This factor informs how well the body is able to produce chemically stored energy from nutrition and to make this energy available.

η=Energy consumption for cell division activity; is a part of the metabolism that is necessary for cell reproduction/cell renewal. If this value is in the “red zone”, it means an increased breakup of cells which actually should have been regenerated.

θ=Regular and irregular neoplasm: Shows, comparable with the above mentioned value, the relation between cell degradation and cell production and indicates a possible disturbance.

ξ=Inflammatory regular neoplasm: Gives particular information on cell neoplasm within the scope of inflammatory processes.

ρ=Health threat risk parameters: A summarizing statement derived from all the other values.

σ=Activated neoplasm: A particular information on neoplasm, an additional value.

φ=Complex trait of metabolism: mirrors everything involved with metabolism.

χ=Redox equivalent, respectively redox potential: Shows the redox state respectively changes of the redox state. Information on the possibility of oxidative stress can be gained, as well as how much of reductive capacity is lost at the moment. The relationship between oxidants and antioxidants is shown, as well as its disturbance, which, after all, suggests disturbances of the bioregulation.

These aforementioned specific state variables Zₙ are depicted as absolute values 26 and as color gradients 27 on the image displaying device 17, as schematically shown in FIG. 11. A “red” value shows an increased activation of the regulation process, which can be interpreted both negatively and positively, for example in case of a necessary activation during an infection. So it is important to view the values of the specific state variables Zₙ as a whole, and not to view them individually.

1. Device for optical examination and for evaluation of a biological active and/or biological activatable substance (2) by use of infra-red, visible or ultra-violet light, at which the examined, respectively evaluated substance is optically stimulated, so that the substance either emits light (in particular fluorescence analysis), or modifies the wavelength of the used light (in particular raman analysis), whereas the device comprises:
an electrically driven light source (3),
an optical light guide (4), by which the light emitted by the light source (3), is transferable as radiation to the substance being examined (2),
an optical or opto-electronical sensor device (5) dedicated to the substance being examined (2), to receive the light emitted or modified by the biological active and/or biological activatable substance,
an optical or opto-electronical spectrometer device (6) dedicated to the sensor device (5), by which the light emitted or modified by the substance (2) is measured regarding to light intensity at not less than one frequency, or regarding to frequency shift, whereas a corresponding measurement signal is passed to an analysis circuitry assigned to the spectrometer device (6), and
the device comprising a measurement surface (11), which in shape and dimensions fits to the biological active and/or biological activatable substance (2) being examined.

2. Device as claimed in claim 1, characterized in that the biological active and/or biological activatable substance (2) is a biological tissue, i.e. a tissue of human, plant or animal origin.

3. Device as claimed in claim 1 or 2, characterized in that at least a portion of the optical light guide (4) and the sensor device (5) are placed in a housing (12), said housing comprising a contact area (13) on or in the upper portion of the housing, with the contact area (13) including the measurement surface (11), or at least partially correlating with the measurement surface (11), on said contact area (13) a part of the body of the substance being examined (2), in particular a hand, a forearm, a leg or a foot is applied, whereas an alignment device (14) is assigned to the contact area (13) on or in the upper portion of the housing for aligning the body part of the test subject with the contact area (13).

4. Device as claimed in claim 1, 2 or 3, characterized in that the optical light guide (4) features a light emission segment (8), which forms, together with the sensor device (5), a structural entity (9), said entity (9) comprising the measurement surface (11) on its upper or outer surface.

5. Device as claimed in one or more of the preceding claims, characterized in that the alignment device (14) comprises a recess (14) or a bulge on or in-the upper portion of the housing (12), the width of said alignment device (14) approximating the width of a middle finger.

6. Device as claimed in one or more of the preceding claims, characterized in that the measurement surface (11) is of almost convex shape following approximately the contours of a slightly clenched hand (2), or the measurement surface (11) being curved strongly convex with the shape following approximately the contours of a clenched hand.

7. Device as claimed in one or more of the preceding claims, characterized in that an image displaying device (17) is placed on the measurement surface (11) or adjacent to it.

8. Device as claimed in one or more of the preceding claims, characterized in that the measurement surface (11) is placed on a measurement head, said measurement head being connected to the light source (3) via the optical light guide (4).

9. Device as claimed in one or more of the preceding claims, characterized in that all optical and/or opto-electronical components such as the light source (3), the optical light guide (4), the sensor device (5) and the spectrometer device (6) are embedded in the housing (12).

10. Device as claimed in one or more of the preceding claims, characterized in that the optical or opto-electronical spectrometer device (6) assigned to the sensor device (5) features a semiconductor sensor apparatus with at least an avalanche photo diode with a band gap corresponding to the frequency or frequency shift to be measured.

11. Device as claimed in claim 10, characterized in that the semiconductor sensor apparatus comprises an adjustable and/or variable frequency gap, with regard to the measurement frequency.

12. Method for optical examination and for evaluation of a biological active and/or biological activatable substance (2) by use of infra-red, visible or ultra-violet light, at which the examined, respectively evaluated substance is optically stimulated, so that the substance either emits light (in particular fluorescence analysis), or modifies the wavelength of the used light (in particular raman analysis), whereas the method comprises the following steps:
irradiation of the substance being examined (2) with light emitted by the light source (3), said light being transferred to the substance (2) via an optical light guide (4),
receiving the light emitted or modified by the biological active and/or biological activatable substance using an optical or opto-electronical sensor device (5) dedicated to the substance being examined,
measurement of the light emitted or modified by the substance (2), regarding to light intensity at not less than one frequency, or regarding to frequency shift, by use of an optical or opto-electronical spectrometer device (6) dedicated to the sensor device (5), and
passing of a corresponding measurement signal to an analysis circuitry.

13. Method as claimed in claim 12, characterized in that the biological active and/or biological activatable substance (2) is a biological tissue, i.e. a tissue of human, plant or animal origin.

14. Method as claimed in claim 12 or 13, characterized in that at least a portion of the optical light guide (4) and the sensor device (5) are placed in a housing (12), said housing comprising a contact area (13) on or in the upper portion of the housing, with the contact area (13) including the measurement surface (11), or at least partially correlating with the measurement surface (11), on said contact area (13) a part of the body of the substance being examined (2), in particular a hand, a forearm, a leg or a foot is applied, whereas an alignment device (14) is assigned to the contact area (13) on or in the upper portion of the housing for aligning the body part of the test subject with the contact area (13).

15. Method as claimed in one or more of the preceding claims, characterized in that by application of the biological active and/or biological activatable substance (2) onto the sensor device (5), different fluorescending components n, m (n, m=a, b, c, d, . . . ) of the substance (2) will be activated by a light impulse emitted by the light source (3), whereupon these components (a, b, c, d, . . . ) start emitting light signals of certain wavelengths and certain intensities (Iₐ, I_{b}, I_{c}, I_{d} etc.), with these light signals (Iₐ, I_{b}, I_{c}, I_{d} etc.) being measured.

16. Method as claimed in claim 15, characterized in that from the measured intensities (Iₐ, I_{b}, I_{c}, I_{d}) of the light signals, multiple, in fact at least three or four, in particular at least five or six different metabolic parameters Sₙ (with n=a, b, c, d, . . . ) are being calculated, whereas the following applies:
Sₙ=F (Iₙ, λₙ)×G (Iₘ, λₘ)
with F and G being two different mathematical functions of intensities and wavelengths, with n, m=a, b, c, d . . . , and n≠m,
and the correlation, respectively the mathematical conjunction X of the functions F and G in the most basic form being a product or a sum, in the following shape
Sₙ=Π[F (Iₙ, λₙ)G (Iₘ, λₘ)] or
Sₙ=Σ[F (Iₙ, λₙ)G (Iₘ, λₘ) ],
with n, m=a, b, c, d, . . . , and n≠m, or n=m.

17. Method as claimed in claim 15 or 16, characterized in that the components n, m with their respective measured emission wavelengths (λₙ, λₘ) and their measured intensities (Iₙ, Iₘ) in particular exhibit the following biochemical substances:
a=ATP (Adenosine triphosphate), and/or
b=GTP (Guanosine triphosphate), and/or
c=FAD (Flavinadenindinucleotide), and/or
d=NADH (Nicotinamide adenine dinucleotide reduced), and/or
e=NADP (Nicotinamide adenine dinucleotide phosphate), and/or
f=Kynurenine, and/or
g=Orotic acid, and/or
h=Thromboxane, and/or
i=Tryptophan.

18. Method as claimed in one of the claims 15 to 17, characterized in that the fluorescence intensities (Iₙ, Iₘ) are measured for the components n, m=a, b, c, d, . . . in the wavelength range (λₙ, λₘ) from 287 to 800 nm, preferably from 340 to 600 nm.

19. Method as claimed in one of the claims 15 to 18, characterized in that measurement of the fluorescence intensities (Iₙ, Iₘ) occurs at a defined moment (tₙ, tₘ) and/or in defined intervals (Δtₙ, Δtₘ) whereas the following applies:
n≠m
tₙ≠tₘ
Δtₙ≠Δtₘ

20. Method as claimed in one of the claims 15 to 19, characterized in that at a defined moment (tₙ, tₘ) of the measurement a psychic or physical stress is applied on the patient, and that the fluorescence intensities (Iₙ, Iₘ) are being measured several times before and after stress application, and that the metabolic regulation is being determined.

21. Method as claimed in one or more of the preceding claims, characterized in that from optical examination or evaluation of a biological active and/or biological activatable substance and from measurement of the metabolic parameters Sₙ (with n=a, b, c, d, . . . ), various different specific state variables Zₙ are being calculated and graphically depicted as mathematical functions of the metabolic parameters Sₙ, whereas these specific state variables Zₙ comprise the following:
α=Cell division activity, and/or
β=Metabolic activity, and/or
γ=Activity of biosynthesis, and/or
δ=Regulation of immune system, and/or
ε=Inflammations, and/or
ζ=Metabolic rate, and/or
η=Energy consumption for cell division activity, and/or
θ=Regular and irregular neoplasm, and/or
ξ=Inflammatory regular neoplasm, and/or
ρ=Health threat risk parameters, and/or
σ=Activated neoplasm, and/or
φ=Complex trait of metabolism, and/or
χ=Redox equivalent, and/or
ψ=Mental stability parameter.

22. Method as claimed in one of the claims 16 to 21, characterized in that the biological active components showing autofluorescence are being activated to emit by application of light with an excitation wavelength of 287 nm to 340 nm, preferably 340 nm, onto the cellular and intercellular area.

23. Method as claimed in one or more of the preceding claims, characterized in that the method is utilized for non-invasive examination of control- and regulation processes of human and animal metabolism, and/or for diagnosis of diseases and for preventive examinations, and/or for routine examinations of occupational groups and athletes with a high exposure to physical and psychic stress, and/or for therapy control, and/or for progress of dialysis- and apheresis treatment and for determining the demand for antioxidants.
